(19) United States
(12) Patent Application Publication
(10) Pub. No.: US 2014/0135623 A1
(43) Pub. Date: May 15, 2014

(54) SYSTEMS AND METHODS FOR X-RAY AND ULTRASOUND IMAGING

(71) Applicant: GENERAL ELECTRIC COMPANY, Schenectady, NY (US)

(72) Inventors: Joseph John Manak, Niskayuna, NY (US); Kai Thomenius, Niskayuna, NY (US); Robert Wodnicki, Niskayuna, NY (US); Scott Zelakiewicz, Niskayuna, NY (US); Ying Fan, Niskayuna, NY (US); Andrea Schmitz, Niskayuna, NY (US); Weston Blaine Griffin, Niskayuna, NY (US)

(73) Assignee: General Electric Company, Schenectady, NY (US)

(21) Appl. No.: 13/677,881

(22) Filed: Nov. 15, 2012

(51) Int. Cl. A61B 8/00; A61B 6/04; A61B 8/13; A61B 6/00; A61B 6/02

(52) U.S. Cl. CPC A61B 8/4416; A61B 6/4417; A61B 6/502; A61B 6/02; A61B 8/13; A61B 8/54; A61B 6/04; A61B 8/42; A61B 8/4483; A61B 6/461; A61B 6/469
USPC 600/427

(57) ABSTRACT

An imaging assembly is provided including a paddle assembly, an X-ray detection unit, an ultrasound module, and a control module. The paddle assembly includes first and second plates that are articulable with respect to each other and configured to receive and compress an object to be imaged. The X-ray detection unit is mounted proximate to at least one of the first and second plates. The ultrasound module is configured to acquire ultrasound information of the object to be imaged and includes an ultrasound transducer articulably mounted to at least one of the first and second plates. The control module is configured to position the ultrasound transducer to scan a region of interest identified using X-ray information received from the X-ray detection unit, while not scanning at least a portion of the object outside of the region of interest.

### BACKGROUND

Various imaging modalities may be used to identify or diagnose medical conditions. For example, mammographic X-ray images may be utilized to identify potential lesions or other abnormalities, such as abnormalities that may be indicative of cancer. Typically, if a potential lesion or other abnormality is identified on a mammographic X-ray image, a follow-up visit is scheduled for an ultrasound exam of the breast to confirm or check the initial diagnosis based on the X-ray image.

Such rescheduling typically involves a delay of days or even weeks between exams. This amount of time can lead to patient anxiety and concern between the examinations. Further, because the exams are conducted at separate visits, and also because compression is typically used for mammographic X-ray exams, it is very difficult to co-register the X-ray image and ultrasound image. Present techniques for performing ultrasound examinations of the breast have additional drawbacks, such as the time associated with such examinations.

Previous attempts to combine X-ray and ultrasound imaging modalities into a single exam or patient visit have also met with a number of drawbacks. For example, previous systems have had high costs associated therewith. In addition to the cost of the systems, the systems typically involve the time and expense to conduct an ultrasound of the entire breast, as well as patient discomfort during the examination.

### BRIEF DESCRIPTION

In one embodiment, an imaging assembly is provided. The imaging assembly includes a paddle assembly, an X-ray detection unit, an ultrasound module, and a control module. The paddle assembly includes first and second plates that are articulable with respect to each other. The first and second plates are configured to receive and compress an object to be imaged. The X-ray detection unit is mounted proximate to at least one of the first and second plates. The ultrasound module is configured to acquire ultrasound information of the object to be imaged. The ultrasound module includes an ultrasound transducer articulably mounted to at least one of the first and second plates. The ultrasound transducer is configured to be positioned to acquire ultrasound information of a selected portion of the object to be imaged. The control module is configured to position the ultrasound transducer to scan a region of interest identified using X-ray information received from the X-ray detection unit, while not scanning at least a portion of the object outside of the region of interest.

In another embodiment, a method is provided. The method includes obtaining an X-ray image of the object using information acquired by the X-ray detection unit. The object has been compressed between first and second plates, wherein at least one of the first and second plates has an X-ray detection unit associated therewith. Further, the method includes identifying a region of interest of the object. The method also includes selectively ultrasound imaging the region of interest of the object while the object remains under compression while not ultrasound imaging at least a portion of the object outside of the region of interest.

In a further embodiment, a tangible and non-transitory computer readable medium is provided including one or more software modules configured to direct a processor to obtain an X-ray image of an object using information acquired by the X-ray detection unit. The object has been compressed between first and second plates, wherein at least one of the first and second plates has an X-ray detection unit associated therewith. Further, the one or more software modules are also configured to direct the processor to control an ultrasound module to selectively ultrasound image the region of interest of the object while the object remains under compression while not ultrasound imaging at least a portion of the object outside of the region of interest.

### BRIEF DESCRIPTION OF THE DRAWINGS

FIG. 1 is a flowchart of an exemplary method for imaging an object in accordance with various embodiments.

FIG. 2 is a schematic view of an imaging assembly in accordance with various embodiments.

FIG. 3 is a view of an imaging system at an initial Cranial Caudal (CC) position in accordance with various embodiments.

FIG. 4 is a view of the imaging system of FIG. 3 at a compression position in accordance with various embodiments.

FIG. 5 is a view of the imaging system of FIG. 3 at a reduced compression ultrasound scanning position in accordance with various embodiments.

FIG. 6 is a view of an imaging system at an initial Mediolateral Oblique (MLO) position in accordance with various embodiments.

FIG. 7 is a view of the imaging system of FIG. 6 at a compression position in accordance with various embodiments.

FIG. 8 is a view of the imaging system of FIG. 6 at a reduced compression ultrasound scanning position in accordance with various embodiments

FIG. 9 is a block schematic diagram of an X-ray imaging system in accordance with various embodiments.

FIG. 10 is a block diagram of an exemplary ultrasound imaging system formed in accordance with various embodiments.

FIG. 11 is a block diagram illustrating a portion of the ultrasound imaging system shown in FIG. 10 in accordance with various embodiments.

FIG. 12 is a view of an imaging system in accordance with various embodiments.

### DETAILED DESCRIPTION

Various embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors, controllers or memories) may be implemented in a single piece of hardware (e.g., a general purpose signal processor or random access memory, hard disk, or the like) or multiple pieces of hardware. Similarly, any programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrases “image” or “reconstructing an image” are not intended to exclude embodiments in which data representing an image is generated, but a viewable image is not. Therefore, as used herein the term “image” broadly refers to both viewable images and data representing a viewable image. However, many embodiments generate, or are configured to generate, at least one viewable image.

Systems formed in accordance with various embodiments provide an imaging system including X-ray and ultrasound modalities. In some embodiments, a pair of opposed plates are articulable with respect to each other to position and/or compress an object, such as a portion of a human anatomy (e.g., a breast) to perform an X-ray scan or exam. Using information obtained via the X-ray scan or exam, one or more portions of interest of the object may be identified for further analysis using ultrasound imaging. In some embodiments, an ultrasound imaging module is associated with one or more of the opposed plates (e.g., mounted thereto or therein) and is controlled to perform an ultrasound scan limited to the portion of interest. Thus, in some embodiments, the time between an initial X-ray scan and a subsequent ultrasound scan may be reduced. Further, in some embodiments, the time required for the ultrasound scan may also be reduced.

Some embodiments provide a mammographic X-ray system having a modified compression paddle that includes and/or accepts a cassette containing an ultrasound probe that can be moved to various locations within the field of view of the X-ray system. For example, a control module utilizing appropriately configured software may analyze an X-ray image and identify potential regions of interest, such as potential lesions. If one or more such regions of interest (e.g., lesions) are identified, the control module may position the ultrasound probe to scan the region(s) of interest. In some embodiments, a cassette containing an ultrasound probe may be positioned between a compression plate and an X-ray detector.

Some embodiments provide for improved co-registration of mammographic X-ray and ultrasound images, for example by acquiring such images at substantially the same time and/or by acquiring such images utilizing a same or similar amount of compression.

A technical effect of at least one embodiment includes reduced numbers of patient visits for breast cancer exams (e.g., breast cancer screening exams). A technical effect of at least one embodiment includes reduced time required to acquire and analyze results of mammographic exams, including ultrasound exams. A technical effect of at least one embodiment includes reduced patient distress and anxiety, for example patient distress and anxiety caused by a time delay between an initial and follow-up examination. A technical effect of at least one embodiment includes reducing cost and length of time for an ultrasound scan. A technical effect of at least one embodiment includes improving facility workflow. A technical effect of at least one embodiment includes reducing errors associated with repositioning. A technical effect of at least one embodiment includes improved co-registration of X-ray and ultrasound images, thereby improving diagnoses and/or reducing time and skill required by a medical professional for analysis of acquired images. A technical effect of at least one embodiment includes improved diagnosis of dense breasts, for example by targeting areas with high anatomical noise. A technical effect of at least one embodiment includes improving sensitivity and/or specificity of breast exams. A technical effect of at least one embodiment is reduced cost for a combination ultrasound/X-ray imaging mammographic examination system.

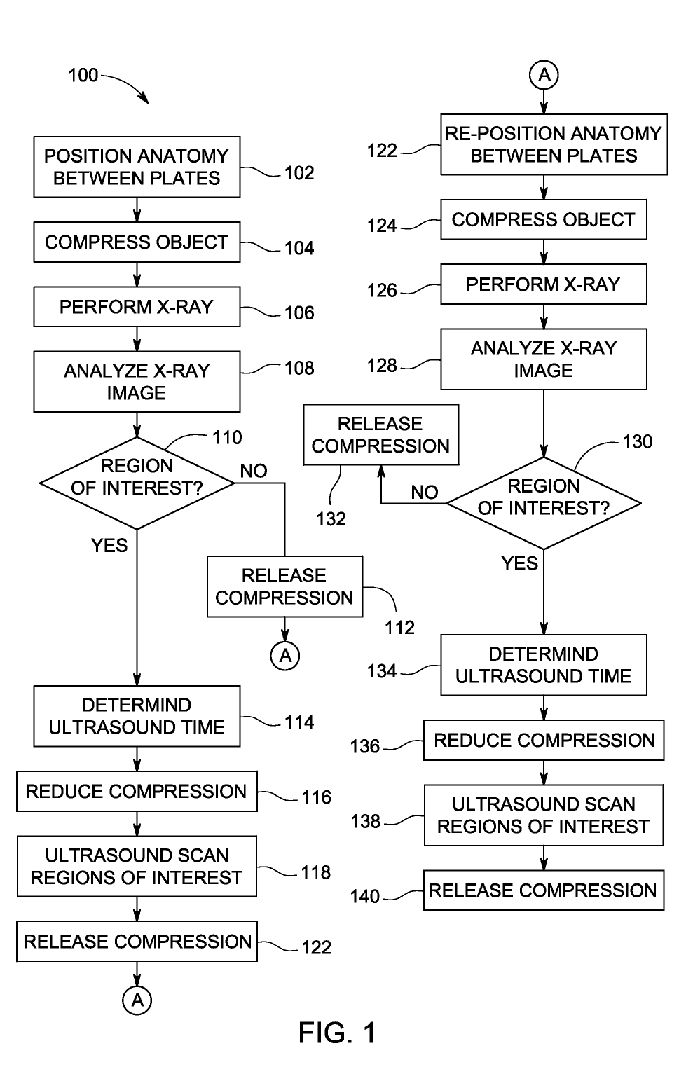

FIG. 1 is a flow chart of a method 100 for imaging an object in accordance with an embodiment. The method 100, for example, may employ structures or aspects of various embodiments discussed herein. In various embodiments, certain steps may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion.

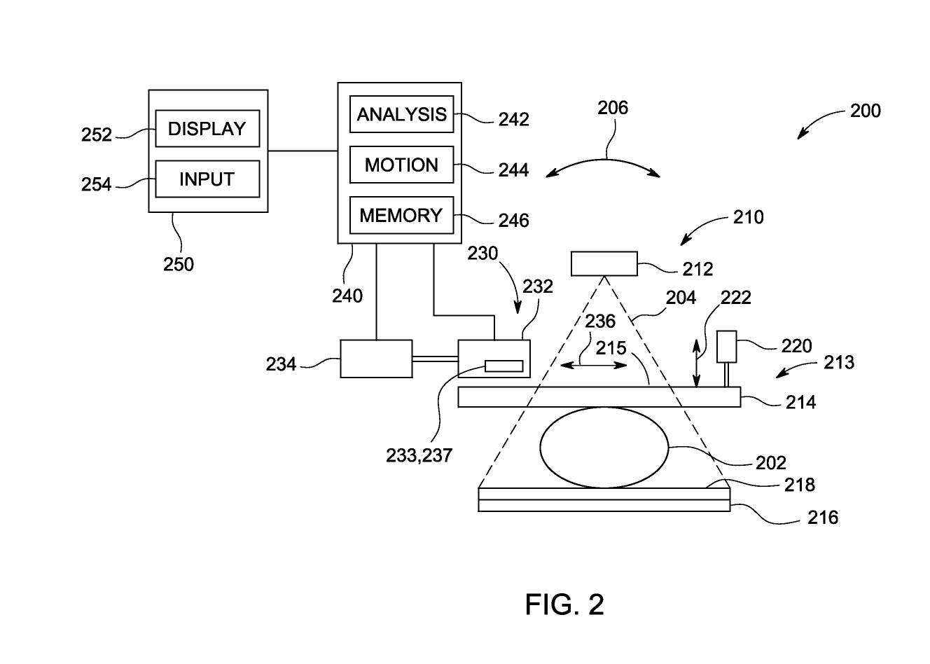

At 102, an object is positioned between plates of an imaging system. For example, the object may be a portion of human anatomy such as a breast. The breast may be positioned to provide, for example, a Cranial-Caudal (CC) view. Because X-ray scans effectively view an object in two dimensions (2D), structures such as breast tissue that exceed a certain density and/or thickness may not be readily amenable to X-ray scans. Compression may be used to render a breast more amenable to X-ray imaging. For example, compression may reduce the thickness of the breast, and stretch tissue to a wider viewing area in two dimensions, allowing for improved identification of structures located during a scan. Further, by presenting a thinner overall structure to an X-ray detecting system, the X-ray dosage required to image the breast may be reduced. FIG. 2 provides an example of an imaging system including opposed plates or paddles that may be brought together to compress a portion of human anatomy or other object.

FIG. 2 is a schematic view of an imaging system 200 formed in accordance with various embodiments. The imaging system 200 is configured to provide both X-ray and ultrasound imaging modalities that may be used to perform scans on an object (e.g. a patient) in the same visit to a scanning location. For example, the imaging system 200 may be used to perform an X-ray scan and an ultrasound scan substantially consecutively. Substantially consecutively may be understood to describe, for example, scans that are performed with a relatively short time interval therebetween. In some embodiments, the X-ray scan and the ultrasound scan may be performed substantially consecutively with a time interval of less than about 5 seconds therebetween. In some embodiments, the X-ray scan and the ultrasound scan may be performed substantially consecutively with a time interval of less than about 10 seconds therebetween. In some embodiments, the X-ray scan and the ultrasound scan may be performed substantially consecutively with a time interval of less than about 1 minute therebetween. In some embodiments, the X-ray scan and the ultrasound scan may be performed substantially consecutively with a time interval of less than about 5 minutes therebetween. However, it should be appreciated that other time intervals are contemplated as well.

The imaging system 200 may be used to image an object, such as a human breast 202. The imaging system 200 may be articulable with respect to the object being imaged. In the illustrated embodiments, the imaging system 200 is articulable in a rotational direction 206 and thus may be used to view the breast 202 from a variety of angles for different scans. For example, a first X-ray scan may be performed at a first angle, and a second X-ray scan may be performed at a second angle to provide a different view of the breast 202. Because the breast 202 is a three dimensional object and the X-ray scan effectively sees the breast 202 in two dimensions, a structure within the breast 202 may be obscured, blocked, or otherwise un-identifiable at one angle or view, but may be identifiable when viewed at a different angle or view. Thus, improved identification of structures within the breast 202 may be achieved by performing X-ray scans (and/or ultrasound scans) at two or more different angles or views.

In the illustrated embodiment, as discussed above, the system 200 is configured to obtain a 2-dimensional X-ray image. In various embodiments, the system 200 may be configured to obtain a 3-dimensional X-ray image, such as via 3-dimensional tomosynthesis. In some embodiments, tomosynthesis imaging information may be acquired utilizing a tube or other structure that may rotate between about 10 and 30 degrees in one or more directions to provide a volumetric image. In some embodiments, the amount of compression applied between plates or paddles may be reduced (e.g., in connection with the use of 3-dimensional tomosynthesis). For example, an amount of compression that is sufficient to position the object (e.g., breast) may be used. Thus, in various embodiments, various imaging techniques may be employed. Further, various mountings of an X-ray detection unit proximate to a plate or paddle may be employed (e.g., stationary or rotational).

The imaging system 200 includes an X-ray module 210, an ultrasound module 230, a control module 240, and an interface 250. Generally speaking, in the illustrated embodiment, the X-ray module 210 is configured to perform an X-ray scan of the object 202, and to provide X-ray imaging information to the control module 240. The control module 240 is configured to analyze the X-ray information (e.g., an X-ray image) and to identify one or more regions of interest (e.g., lesions or other aspects for which follow-up examination and/or additional scans may be desired). The identification of one or more regions of interest, in some embodiments, includes one or more user inputs. The control module 240 is configured to control the ultrasound module 230 to perform an ultrasound scan of the one or more regions of interest to facilitate improved analysis and/or diagnosis of the one or more regions of interest. For example, the ultrasound scan may be used to confirm whether or not one or more regions of interest were false positives in the X-ray scan (e.g., not a cause for concern) or whether not one or more regions of interest appear to be of medical interest (e.g., potentially cancerous). In various embodiments, the control module 240 may select one or more regions autonomously. In some embodiments, one or more regions of interest may be selected by a practitioner via the interface 250, for example, by selecting one or more regions using an image displayed by the interface 250.

The X-ray module 210 includes an X-ray source 212, a paddle assembly 213 (including an upper plate 214 and a lower plate 216), a detector 218, and an actuator 220. The X-ray source 212 is configured to emit X-rays that pass through an object (e.g., object 202) and are received by the detector 218. The detector is position on, mounted to, and/or forms a part of the lower plate 216. Information acquired by the detector 218 is communicated to the control module 240. The X-ray source 212 in the illustrated embodiment has a field of view 204 that projects on to the detector 218.

The paddle assembly 213 includes an upper plate 214 and lower plate 216. The upper plate 214 and lower plate 216 are an example of first and second opposed plates that are articulable with respect to each other. In the illustrated embodiment, the lower plate 216 is fixed and the upper plate 214 is articulable along a compression direction 222 by the actuator 220. The upper plate 214 may be articulated downward (in the sense of FIG. 2) toward the lower plate 216 to compress the breast and upward away from the lower plate 216 to reduce an amount of compression on the breast 202 and/or to release the breast 202 from between the upper plate 214 and the lower plate 216. In alternate embodiments, other arrangements may be employed to provide articulation of two plates with respect to each other. For example, in some embodiments, both plates may be movable, while in other embodiments, the upper plate may be fixed and the lower plate movable. In the illustrated embodiment, the upper plate 214 and the lower plate 216 are depicted as substantially flat. In alternate embodiments, plates may be employed having curved or otherwise contoured profiles. Other types or orientations of articulation may be employed as well. As one example, in some embodiments, the first and second plates may be coupled by a pivot and thus be rotatable with respect to each other. The actuator 220 may be controlled by the control module 240 and/or an operator. In various embodiments, a variety of devices or mechanisms (e.g., one or more motors, pneumatic or hydraulic cylinders, electronic linear actuators, hand-operated mechanisms, or the like) may be employed to articulate the plates. In some embodiments, one or more paddles or plates may translate and/or rotate on a gantry which may be mounted to a floor and/or a wall.

In various embodiments, the upper plate 214 and/or the lower plate 216 may be configured to reduce any potential attenuation (e.g., radiolucent) of an X-ray as the X-ray passes through the plates. Further, in various embodiments, the upper plate 214 and/or the lower plate 216 may be substantially transparent to provide an operator with visual confirmation of the positioning of the object 202.

The detector 218 is configured to receive X-ray beams that have been emitted from the X-ray source 212 and have passed through the breast 202, and to provide X-ray imaging information to the control module 240. The control module 240 is configured to receive the X-ray image information from the detector 218 and/or to reconstruct an X-ray image using the X-ray information from the detector 218. In some embodiments, the detector 218 may include more than one detector, such as an array of detectors. In the illustrated embodiment the detector 218 is mounted to the lower plate 216. In other embodiments, the detector 218 may be a part of, embedded within or otherwise associated with a plate or paddle.

The ultrasound module 230 is configured to acquire ultrasound information of the object to be imaged. In the illustrated embodiment, the ultrasound module 230 includes an ultrasound transducer 232, a dispensing module 233, an actuator 234, and a reservoir 237. The ultrasound transducer 232 is configured to send an ultrasonic beam or beams through a portion of an object and to receive returned ultrasonic beams. Information acquired by the ultrasound transducer is then used to reconstruct an image corresponding to the portion of the object scanned. For example, information from the ultrasound transducer 232 may be communicated to the control module 240 and/or the interface 250 for image reconstruction and/or analysis.

In some embodiments, the ultrasound transducer 232 includes an array of aligned transducers that are configured to be articulated in a substantially lateral direction, allowing for a region of interest of the breast to be ultrasonically scanned in a single pass. The ultrasound transducer 232 may be part of a cassette type assembly that is movable within and/or along a plate or paddle (as one example, an upper surface 215 of the upper plate 214, or, as another example, a lower surface of the lower plate 216). A liquid or gel may be employed to create or improve an acoustic contact between the ultrasound probe and a casing or surface of the plate or paddle.

In some embodiments, to speed up acquisition of an ultrasound image, Multi-Line Acquisition (MLA) may be performed in which a single broad transmit beam is used to insonify a region of the breast 202 encompassing multiple axial receive beams. The resulting echoes, for example, may then be processed through specialized beamforming electronics which are capable of generating multiple simultaneous receive beams from the received data. Various embodiments include multiple simultaneously operating probe heads. The probe heads may be spaced a sufficient distance apart to minimize acoustic cross-talk. Additionally, orthogonal excitation codes may be used so that cross-talk on adjacent probe heads is minimized in the beamformer. Further still, in some embodiments, the ultrasound probe may include a two dimensional array capable of steering the beam in order to facilitate spatial compounding. In some embodiments the ultrasound probe may be configured as a mechanically scanned static annular array or an electronically scanned reconfigurable annular array to reduce the number of beamforming channels used, thereby reducing cost and complexity of the system. Yet further still, in some embodiments, a control module may be configured to determine the outline of the breast 202 during acquisition, with the outline information used in real-time during mechanical scanning of the probe head to reduce ultrasound acquisition time by only scanning regions of the plate or paddle where the breast 202 is known to be located.

The actuator 234 is configured to articulate the ultrasound transducer 232 to a desired position for scanning a region of interest of the object (e.g., a region of interest of the breast 202). The actuator 234 may position the ultrasound transducer based on control signals or messages received from the control module 240. In the illustrated embodiment, the actuator 234 is configured to articulate the ultrasound transducer 232 in an ultrasound direction 236 substantially laterally along an upper surface 215 of the upper plate 214. In various embodiments, the actuator 234 may include one or more of a variety of devices or mechanisms (e.g., one or more motors, pneumatic or hydraulic cylinders, electronic linear actuators, or the like).

The ultrasound transducer 232 may be positioned outside of the field of view 204 of the X-ray source 212 while an X-ray scan is being performed. After the X-ray scan is complete and a region of interest has been selected, the actuator 234 may position the ultrasound transducer 232 to scan the region of interest. Thus, the ultrasound transducer 232 may be articulable between a position outside of the field of view 204 and one or more positions inside of the field of view 204. In some embodiments, the ultrasound transducer may be mounted to one or more paddles and plates, and articulable across one or more surfaces, for example, via a track or guide. In some embodiments, the ultrasound transducer may be movable in a plurality of lateral directions (e.g., the actuator 234 may include a plurality of linear actuators or otherwise be configured to articulate the ultrasound transducer 232 in a plurality of directions). For example, the actuator 234 may be configured to move the ultrasound transducer in a raster pattern sized and configured to cover a region of interest. Further still, in some embodiments, the ultrasound transducer may be removably mounted to a paddle or plate, and physically removed from the paddle or plate during X-ray scanning.

The dispensing module 233 in the illustrated embodiment illustrated in FIG. 1 includes a reservoir 237 (e.g., a sealed reservoir). The dispensing module 233 is configured to dispense a liquid or gel from the reservoir 237 to acoustically couple an ultrasound transducer with a surface of a plate or paddle. For example, in the illustrated embodiment, the dispensing module 233 is configured to dispense a liquid to the upper surface 215 of the upper plate 214 over which the ultrasound transducer 232 traverses during ultrasound scanning of one or more regions of interest. The liquid or gel is configured to improve the acoustic contact between a transducer and a plate or paddle, so that soundwaves may be transmitted between the transducer and the object via the plate or paddle (e.g., with the plate or paddle pressed against the object to be scanned as the object is compressed). In some embodiments, a portion of the dispensing module and/or a surface of a plate or paddle may be configured to improve retention of liquid or gel on the surface when the imaging system 200 is articulated in the rotational direction 206 at an angle in which gravity may urge the liquid or gel off of the surface (e.g., a position as depicted in FIG. 8).

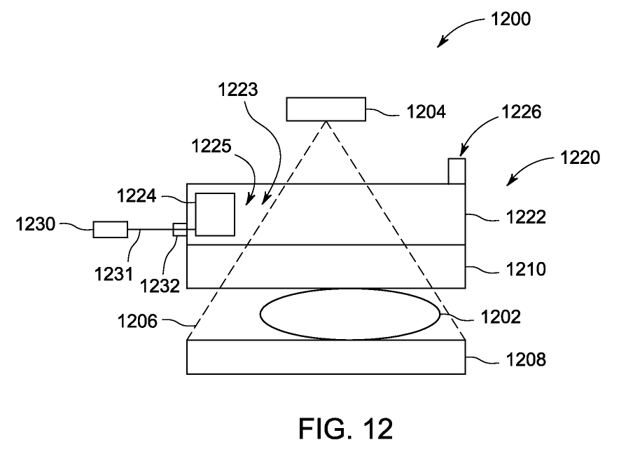

In other embodiments, a liquid or gel may be retained in a housing or structure through which an ultrasound transducer passes. FIG. 12 provides a view of a system 1200 including a liquid or gel filled housing through which an ultrasound transducer travels. The system 1200 include an emitter 1204 that emits X-rays having a field of view 1206. The system 1200 may be used, for example, to image an object 1202. The object 1202 is compressed between an upper plate 1210 and a lower plate 1208 during X-ray imaging.

The system 1200 also includes an ultrasound assembly 1220. The ultrasound assembly 1220 includes a housing 1222, a transducer 1224, and a port 1226. The housing 1222, for example, may be mounted to a plate or paddle, mounted proximate to a plate or paddle, or incorporated into a plate or paddle. The housing 1222 has a generally hollow interior configured to retain a material 1225 such as a liquid or gel for acoustically coupling the transducer 1224 to a surface in direct or indirect contact with the object 1202 (e.g., a bottom surface of the housing 1222). In some embodiments, the housing 1222 is configured to provide a sealed volume 1223 within an interior portion of the housing 1222, with the material 1225 contained within the sealed volume 1223, and the transducer 1224 configured to be disposed within the sealed volume 1223 and to be articulated through the sealed volume 1223. The housing 1222 may also include, for example, rails, tracks, or the like within an interior portion for guiding the transducer 1224 as an actuator 1230 articulates the transducer.

In some embodiments, at least a portion of the actuator 1230 may be disposed external to the housing 1222 containing the material 1225 (e.g., acoustic coupling fluid). For example, the actuator 1230 may include an output shaft 1231 that extends through the housing 1222 via a shaft seal 1232 that is configured to prevent or minimize any fluid leakage, but allow the shaft 1231 to rotate relatively easily. In some embodiments, more than one actuator may be employed to articulate the transducer 1224 in more than one direction. The output shafts of the actuators may, for example, be coupled to a cable drive system to move the transducer 1224 with two degrees of freedom. In one embodiment, the cable drive system may be configured to drive a transducer mounted to an orthogonal rail system to provide capability for a Cartesian scanning of the object 1202. Furthermore, the cable drive system may be configured such that the two axes of motion of the Cartesian system (X and Y) may be controlled independently by two external actuators, thereby providing a decoupled motion control system.

As seen in FIG. 12, the ultrasound transducer 1224 may be positioned outside of the field of view 1206 during emission of a X-ray beam and acquisition of X-ray imaging information. Further, in some embodiments, during X-ray imaging, all or a portion of the material 1225 may be evacuated from the housing 1222 via port 1226 to protect the material 1225 and/or improve X-ray imaging. After X-ray imaging information has been obtained, the material 1225 may be re-introduced into the housing 1222 and the transducer 1224 positioned to obtain ultrasound information of a region of interest identified using X-ray information.

Returning to FIG. 2, the control module 240 is configured to position the ultrasound transducer to scan a region of interest identified using X-ray information received from the detector 218. In some embodiments, the control module may be configured to autonomously select one or more regions of interest. For example, the control module may include or have access to software that facilitates the identification of lesions or other regions of interest in an X-ray image. In some embodiments, the control module 240 may receive an input from a practitioner identifying one or more regions of interest, and the control module 240 may, responsive to the input, control the ultrasound module 230 to scan the one or more regions of interest. The control module 240 in the illustrated embodiment includes an analysis module 242, a motion control module 244, and a memory 246.

The analysis module 242 is configured to receive information from the detector 218 of the X-ray module 210 and the ultrasound transducer 232, and to reconstruct images using the information. The analysis module 242 may also be configured, for example, to adjust or account for compression when reconstructing an image using ultrasound information from the ultrasound transducer 232. In some embodiments, reconstructed X-ray and/or ultrasound images may be provided by the control module to a practitioner or other system via the interface 250. In the illustrated embodiments, the analysis module 242 is also configured to identify one or more regions of interest based on X-ray information obtained from the detector 218. For example, in some embodiments, the analysis module 242 is configured to autonomously identify one or more potential lesions or other aspects of interest based on X-ray information received from the detector 218. In some embodiments, the region or regions of interest may be identified by a practitioner based on an analysis of an X-ray image.

Further, in some embodiments the analysis module 242 is configured to determine or estimate an amount of time that will be consumed by an ultrasound scan of the selected or otherwise identified region or regions of interest. One or more settings of the imaging system 200 may be adjusted if the determined time exceeds a predetermined threshold. Further still, one or more ultrasound parameters may be changed based on thickness and/or density of a breast as determined by information obtained during an X-ray scan and/or determined from an analysis of an image. For example, if the determined time for the ultrasound scan exceeds a threshold, the upper plate 214 and lower plate 216 may be separated to reduce the amount of compression on the breast 202 to increase patient comfort. However, if the determined time is less than the threshold, then the amount of time under the original amount of compression (e.g., the amount of compression used during the X-ray scan) may be maintained. Thus, the threshold may be selected based on an amount of time a typical patient may experience a tolerable or acceptable amount of discomfort due to compression. Further still, the amount of reduction in pressure may be proportional to the amount of time determined, with a larger amount of reduction in compression employed for longer scans. In other embodiments, the threshold may be adjustable, for example, on a patient-by-patient basis based on individual tolerances for discomfort due to compression. The movement of the plates to reduce the compression may be performed by an operator or practitioner, for example, in response to a prompt provided by the control module 240, or may be performed autonomously by the imaging system 200. If the analysis module 242 determines that there are no regions of interest that merit examination by ultrasound, the control module 240 may control the upper plate 214 and the lower plate 216 to separate a sufficient amount to remove all compression and release the breast 202 from between the upper plate 214 and the lower plate 216.

The motion control module 244 is configured to control movement and/or position of the plates 214, 216 and/or the ultrasound transducer 232. In various embodiments, the motion control module 244 may be configured to issue control commands to draw the upper plate 214 and lower plate 216 together to provide compression, to release the plates 214, 216 from compression if no region of interest for ultrasound scanning is identified, and/or to reduce an amount of compression between the plates 214, 216 for an ultrasound scan (e.g., to reduce an amount of compression based on a time determined for performing the ultrasound scan). The motion control module 244 is also configured to position the ultrasound transducer 232. In the illustrated embodiments, the motion control module 244 is configured to articulate the ultrasound transducer (e.g., via a control command sent to the actuator 234) from an initial position outside of the field of view 204 to a scanning position proximate an identified region of interest. In some embodiments, the motion control module 244 may control movement of the ultrasound transducer 232 proximate the region of interest (e.g., in a raster pattern), while in some embodiments, the motion control module 244 may maintain the ultrasound transducer 232 in a desired position while an ultrasound beam is manipulated or moved electronically. Thus, a region of interest may be identified using an X-ray image, and the ultrasound transducer 232 may be positioned to scan the region of interest instead of the entire breast 202, thus saving time and money for the scanning process as well as reducing or eliminating patient discomfort.

The interface 250 is configured to allow information and/or commands to be communicated between the control module 240 and a practitioner. In the illustrated embodiments, the interface 250 includes a display module 252 and an input module 254. The display module 252 may include, for example, a printer, a screen, a touchscreen, a speaker, or the like. The input module 254 may include a touchscreen (e.g., a touchscreen shared between the display module 252 and the input module 254), a mouse, stylus, keyboard, keypad, or the like. One or more reconstructed images may be displayed via the display module 252.

The input module 254 is configured to receive input from a practitioner to perform one or more imaging activities. For example, the input module 254 may receive input from a practitioner establishing one or more settings or parameters for imaging. Further, the input module 254 may receive input from a practitioner establishing a region of interest for ultrasound scanning or providing information from which a region of interest may be determined. For example, in some embodiments, the interface 250 may display an X-ray image to a practitioner, and the practitioner may identify and select a region of interest (e.g., using one or more of a stylus, touchscreen, cursor, or the like) for ultrasound scanning. As another example, the practitioner may select a lesion or aspect of interest using the input module 254, and the control module 240 may then determine an appropriate region of interest for ultrasound scanning (e.g., by adding a buffer zone and/or compensating or accounting for a change in compression between X-ray and ultrasound scans). As yet another example, the control module 240 may be configured to identify one or more lesions or aspects of interest. The selected one or more lesions or aspects may be displayed to a practitioner by the interface 250, with the practitioner using the input module 254 to confirm that one or more of the lesions or aspects should be ultrasound scanned and/or identifying one or more regions of interest surrounding or corresponding to the identified lesions or aspects. For example, one or more lesions may be labeled on a display presented to the practitioner along with prompts requesting whether or not each particular labeled lesion should be ultrasound scanned on an individual basis.

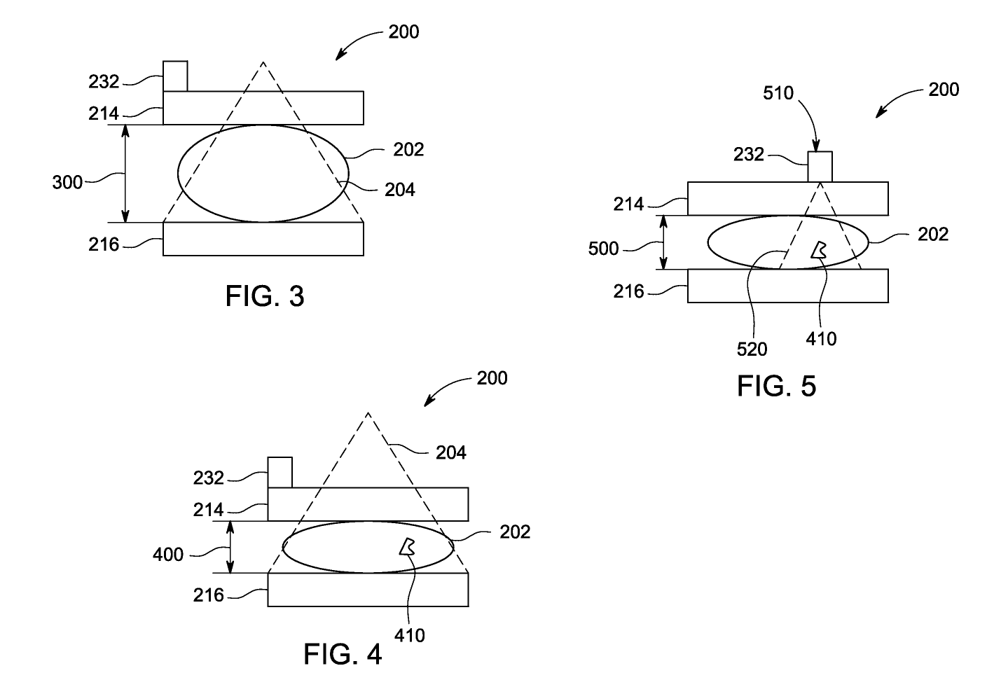

FIG. 3 provides a view of the imaging system 200 at an initial position (e.g. a position corresponding to step 102 of FIG. 1) in accordance with various embodiments. The initial position as depicted in FIG. 3 is with plates near to but not compressing the breast 202, and with the imaging system 200 oriented to obtain a CC view. The upper plate 214 and the lower plate 216 are separated by an initial distance 300. The ultrasound transducer 232 is positioned outside of the field of view 204 so that the ultrasound transducer 232 does not affect and/or is not affected by X-ray beams emitted by the X-ray source 212 (see FIG. 2).

Returning to FIG. 1, at 104, the object (e.g., breast 202) is compressed. Compression of the breast 202 may result in improved imaging and/or a reduction in X-ray dose. For example, compression may provide an improved view of tissue by spreading out the breast 202 and reducing shadows and/or obscuring of portions of tissue by other tissue, provide for more efficient passage of X-rays through a thinner cross-section, and/or reduce the strength of an X-ray beam required to image the breast 202. The object may be compressed between opposed plates or paddles (e.g., upper plate 214 and lower plate 216) that are articulable with respect to each other (e.g., via actuator 220). In some embodiments, one plate or paddle may be fixed while the other plate or paddle articulates in one or more directions. In other embodiments, both plates or paddles may be movable. The opposed plates or paddles may be drawn together to compress the object. An example of compression is depicted in FIG. 4.

FIG. 4 provides a view of the imaging system 200 at a compression position in accordance with various embodiments. The compression position as depicted in FIG. 4 is with plates drawn nearer to each other so that the object (e.g., breast 202) is compressed for improved X-ray imaging. In FIG. 4, the upper plate 214 and the lower plate 216 are separated by a compression distance 400 that is less than the initial distance 300. The ultrasound transducer 232 remains positioned outside of the field of view 204 so that the ultrasound transducer 232 does not affect and/or is not affected by X-ray beams emitted by the X-ray source 212 (see FIG. 2). With the breast 202 compressed between the plates 214, 216, an X-ray scan may be performed, and a lesion 410 may be identified.

Returning to FIG. 1, at 106, an X-ray scan is performed. An X-ray beam may be emitted from an X-ray source (e.g., X-ray source 212), pass through an object (e.g., breast 202), and be received by a detector (e.g., detector 218). Information acquired at the detector may be used in reconstructing an X-ray image. For example, an X-ray image may be reconstructed by an analysis module (e.g., analysis module 242 of control module 240). In some embodiments, the X-ray image may be provided via a display (e.g., display module 252 of interface 250) for viewing by a practitioner.

At 108, the X-ray image is analyzed. In some embodiments, an X-ray image of the breast 202 is examined for lesions or other aspects of interest, such as portions of the image that may correspond to cancerous growths for which examination by ultrasound may be desired. The X-ray image may be analyzed by a practitioner and/or by an analysis module (e.g., analysis module 242 of the control module 240). For example, in some embodiments, the interface 250 may be configured to present an X-ray image representative of the breast 202 to a practitioner for analysis. In some embodiments, an analysis module may analyze the X-ray image and autonomously identify lesions or other aspects of interest. In some embodiments, the analysis module may autonomously identify lesions or other aspects of interest, with the identified lesions or other aspects of interest labeled or otherwise identified on an image presented to a practitioner. For example, with reference to FIG. 4, a lesion 410 (or other aspect of interest for which further examination is desired) may be identified by examination of an X-ray image. The region of interest identified for ultrasound scanning may be determined based on the size and position of the lesion 410. For example, a buffer or margin of error may be employed such that the region of interest identified is larger than the lesion 410 to ensure the entire lesion is scanned. The region of interest may also be sized and positioned to account for potential shifting or changing of size or position of the lesion if compression is reduced for the ultrasound scan.

Returning to FIG. 1, at 110, it is determined if a region of interest for further ultrasound scanning has been identified. The determination may be made autonomously by a control module (e.g., control module 240) in some embodiments and by a practitioner (e.g., responding to a prompt) in some embodiments. If a region of interest has not been identified, the method 100 may proceed to 112, where the plates are separated to release compression on the breast 202. In some embodiments, the release of compression may be performed autonomously once a control module has determined that an ultrasound scan is not to be performed. If there are one or more regions of interest to be ultrasound scanned, the method 100 proceeds to 114.

At 114, an ultrasound scanning time is determined. The time may be determined autonomously by a control module (e.g. analysis module 242 of control module 240). In some embodiments, the control module may first determine a total number of regions of interest, estimate a time for scanning each region of interest, and then add the individual estimated times to determine a total ultrasound scanning time.

At 116, if the determined ultrasound scanning time exceeds a threshold, compression between the plates on the breast 202 is reduced. For example, the plates may be separated an amount such that compression is still maintained on the breast 202, but at a reduced amount compared to the compression used during X-ray scanning. If the scanning time is determined to be less than the threshold, compression may be maintained at the same amount as used during X-ray scanning. Identification of a region of interest for scanning instead of ultrasound scanning the entire breast 202 may achieve substantial time savings. For example, in embodiments where regions of interest are identified autonomously, a control module may identify a region of interest relatively quickly, for example in about 1 second or less. If a region of interest is relatively small, the ultrasound scan may similarly take a relatively small amount of time to perform, for example, less than about 5 seconds, or as another example, less than about ten seconds. Thus, the time to analyze an X-ray image and ultrasound scan a region of interest may take seconds instead of the about ten minutes that may be required to ultrasound the entire breast 202. In some embodiments, the amount of reduction of compression may be proportional to the time for ultrasound scanning (e.g., less compression for longer times). In alternate embodiments, compression may be reduced without respect to estimated scanning time, maintained without respect to estimated scanning time, released without respect to estimated scanning time, or reduced if the estimated scanning time exceeds a first threshold and released if the estimated scanning time exceeds a second threshold. Generally speaking, the closer the amount of compression used is between the X-ray and the ultrasound scans, the less difficulty there will be in co-registering or otherwise comparing the images. Maintenance of at least some of the compression from the X-ray scanning during the ultrasound scanning helps maintain the breast 202 in a similar position, improving co-registration of the images.

At 118, the identified region (or regions) of interest are scanned via ultrasound (e.g., by ultrasound module 230). The selected region (or regions) of interest may define a smaller area or volume than the object being scanned and/or a portion of the object within the field of view 204, thereby reducing the time for an ultrasound scan. An ultrasound probe or transducer may be positioned responsive to commands from a control module proximate the region of interest, with information acquired during the scan communicated to the control module or other system for image reconstruction. The image may be analyzed to check or confirm the initial results or diagnosis obtained via analysis of the X-ray image.

FIG. 5 provides a view of the imaging system 200 at an ultrasound scanning position in accordance with various embodiments. The ultrasound scanning position as depicted in FIG. 5 is with plates drawn somewhat nearer to each other so that the object (e.g., breast 202) is compressed for consistency and improved co-registration with a previously obtained X-ray image, but separated somewhat further than the compression position of FIG. 4 to improve patient comfort during an ultrasound scan that exceeds a time threshold. In FIG. 5, the upper plate 214 and the lower plate 216 are separated by an ultrasound distance 500 that is more than the compression distance 400 but less than the initial distance 300. Thus, the breast 202 remains under compression, but at a lower amount of compression than employed during the X-ray scan. In FIG. 5, the ultrasound transducer 232 is depicted at a scanning position 510 corresponding to a region of interest 520 to be scanned. The position of the ultrasound transducer 232 may be controlled by a control module (e.g., control module 240).

The region of interest 520 in the illustrated embodiment is positioned and sized so that the lesion 410 is within the region of interest 520. For example, the region of interest 520 may be positioned and sized based on the position of the lesion 410 along with a margin of error to provide a buffer to ensure that the entire lesion 410 is scanned as well as to account for any change in size or position of the lesion 410 due to the reduction of compression in the ultrasound scanning position from the compression position depicted in FIG. 4. The region of interest may be selected by a practitioner (e.g., by selecting an area surrounding a lesion or aspect of interest identified on an interface), by a control module responsive to a lesion or aspect of interest identified by a practitioner, or by a control module based on a lesion or aspect of interest identified by the control module. With the ultrasound scan performed with the breast 202 in a similar position and/or under similar compression (or, in the case where compression has not been reduced, the same compression) to that for the X-ray scan, improved co-registration is achieved in comparison to typical exams taken at different visits, with the X-ray performed under compression and the ultrasound performed without compression. Information obtained during the ultrasound scan may be sent to a control module (e.g., control module 240) for further image reconstruction and/or analysis.

Returning to FIG. 1, at 120 the compression between the plates is released so that the breast 202 may be removed and/or repositioned between the plates.

In the illustrated embodiment, at 122, the imaging system (e.g. imaging system 200) is re-positioned (e.g., rotated in a rotational direction 206) to provide a different view of the breast 202. For example, the imaging system may be re-positioned to obtain a Mediolateral Oblique (MLO) view of the breast 202. The MLO view may be used to provide a better view of certain tissue that may not be seen as well via the CC view (e.g. tissue more closely located to the armpit) and/or to provide a different angle of view so that tissue that was shadowed or otherwise obscured in the CC view may be better imaged.

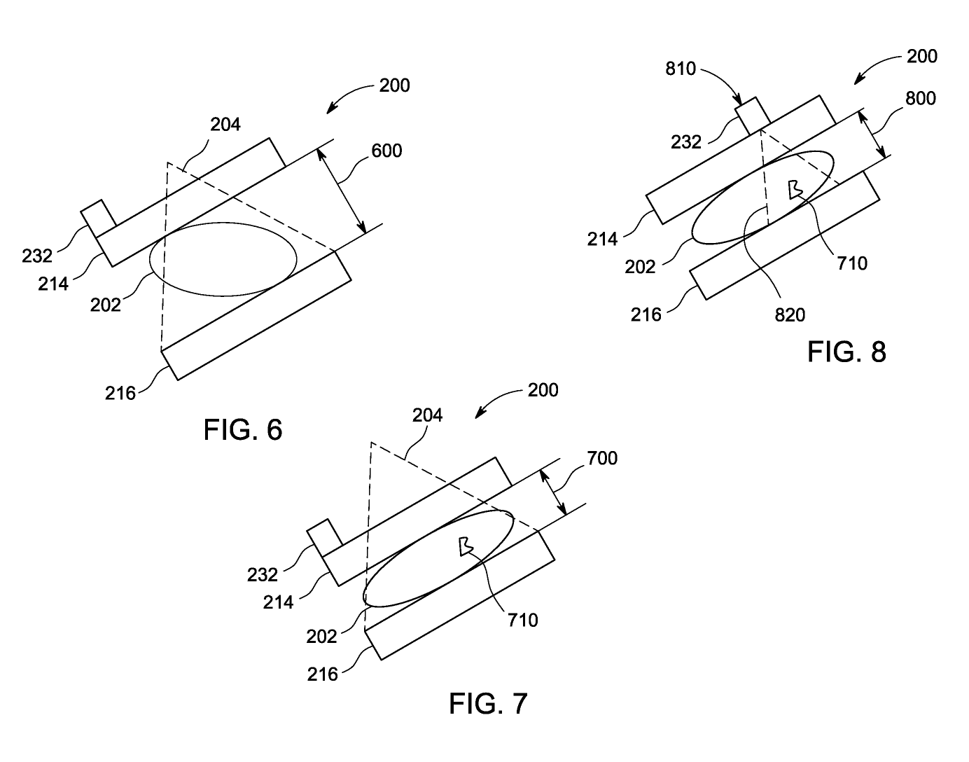

FIG. 6 provides a view of the imaging system 200 at a second initial position (e.g. a position corresponding to step 122 of FIG. 1) to provide an ML) view of the breast 202 in accordance with various embodiments. The position as depicted in FIG. 6 is with plates near to but not compressing the breast 202, and with the imaging system 200 oriented to obtain a MLO view. The upper plate 214 and the lower plate 216 are separated by an initial distance 600. The ultrasound transducer 232 is positioned outside of the field of view 204 so that the ultrasound transducer 232 does not affect and/or is not affected by X-ray beams emitted by the X-ray source 212 (see FIG. 2).

Returning to FIG. 1, at 124 the plates are brought together to compress the breast 202. (See also discussion regarding step 104). FIG. 7 provides a view of the imaging system 200 at a compression position in accordance with various embodiments. The compression position as depicted in FIG. 7 is with plates drawn nearer to each other so that the object (e.g., breast 202) is compressed for improved X-ray imaging. In FIG. 7, the upper plate 214 and the lower plate 216 are separated by a compression distance 700 that is less than the initial distance 600. The ultrasound transducer 232 remains positioned outside of the field of view 204 so that the ultrasound transducer 232 does not affect and/or is not affected by X-ray beams emitted by the X-ray source 212 (see FIG. 2). With the breast 202 compressed between the plates 214, 216, an X-ray scan may be performed, and a lesion 710 may be identified. (See also discussion below.)

Returning to FIG. 1, at 126 an X-ray scan is performed. (See also discussion regarding step 106). At 128, an X-ray image is analyzed. (See also discussion regarding step 108).

At 130, it is determined if a region of interest for further ultrasound scanning has been identified based on the X-ray information obtained at the MLO view. The determination may be made autonomously by a control module (e.g., control module 240) in some embodiments and by a practitioner (e.g., responding to a prompt) in some embodiments. If a region of interest has not been identified, the method 100 may proceed to 132, where the plates are separated to release compression on the breast 202. In some embodiments, the method may proceed to acquire additional images at additional views by re-positioning the imaging system. In some embodiments, the release of compression may be performed autonomously once a control module has determined that an ultrasound scan is not to be performed. If there are one or more regions of interest to be ultrasound scanned, the method 100 proceeds to 134.

If one or more regions of interest to be scanned via ultrasound are identified, at 134 an ultrasound scanning time is determined (see also discussion regarding step 114), and, at 136, an amount of compression may be reduced based on the time determined at 134 (see also discussion regarding step 116 above). At 138, an ultrasound scan of the region or regions of interest is performed. (See also discussion of step 118 above).

FIG. 8 provides a view of the imaging system 200 at an ultrasound scanning position in accordance with various embodiments. The ultrasound scanning position as depicted in FIG. 8 provides a MLO view with plates drawn somewhat nearer to each other so that the object (e.g., breast 202) is compressed for consistency and improved co-registration with a previously obtained X-ray image, but separated somewhat further than the compression position of FIG. 7 to improve patient comfort during an ultrasound scan that exceeds a time threshold. In FIG. 8, the upper plate 214 and the lower plate 216 are separated by an ultrasound distance 800 that is more than the compression distance 700 but less than the initial distance 600. Thus, the breast 202 remains under compression, but at a lower amount of compression than employed during the X-ray scan. In FIG. 8 the ultrasound transducer 232 has been moved to a scanning position 810 corresponding to a region of interest 820 to be scanned. The position of the ultrasound transducer 232 may be controlled by a control module (e.g., control module 240). Information obtained during the ultrasound scan may be forwarded to a control module and/or other system for reconstruction, analysis, and/or diagnosis.

At 140, with the ultrasound scan complete, the compression between the plates is released. In some embodiments, the method may proceed to acquire additional images at additional views by re-positioning the imaging system and proceeding similar to the above discussion.

Thus, embodiments provide methods and systems for maintaining an object (e.g., breast 202) in a same or similar position and/or under a same or similar amount of compression for both an X-ray and an ultrasound scan, with the respective scans taken proximate each other temporally. Such imaging may provide reduced cost (including cost of equipment, cost of time performing scans, and administrative costs associated with the number of patient visits), improved ease of co-registration, improved accuracy of diagnosis, increased patient comfort, and/or reduced patient anxiety.

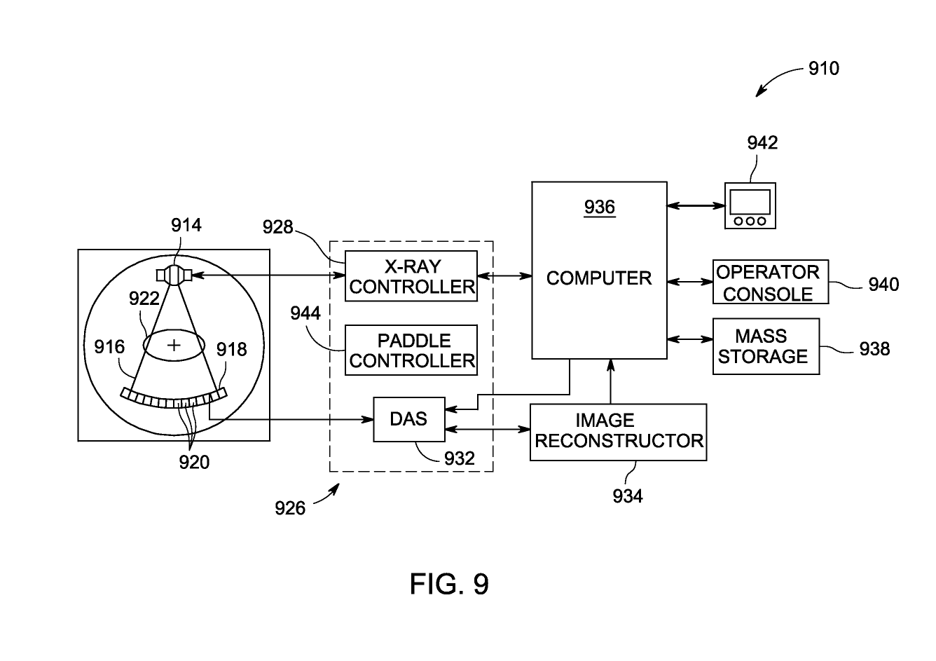

FIG. 9 provides a block schematic diagram of an X-ray system 910 formed in accordance with various embodiments. The imaging-ray system 910 includes an X-ray source 914 configured to project a beam of X-rays 916 toward a detector array 918 positioned opposite the X-ray source 914. In the illustrated embodiment, the detector array 918 is formed by a plurality of detectors 920 which together sense the projected X-rays that pass through an object to be imaged, such as a breast 922. For example, a detector such as detector 218 discussed above may include the detector array 918. While the imaging-ray system 910 is described in connection with FIG. 9 with reference to the breast 922, it should be noted that the X-ray system 910 may have additional applications.

Operation of the X-ray source 914 is governed by a control mechanism 926 of the X-ray system 910. The control mechanism 926, for example, may be incorporated into a control module such as the control module 240. The control mechanism 926 includes an X-ray controller 928 that provides power and timing signals to the X-ray source 914. A data acquisition system (DAS) 932 in the control mechanism 926 samples analog data from the detectors 920 and converts the data to digital signals for subsequent processing. An image reconstructor 934 receives sampled and digitized X-ray data from the DAS 932 and performs high-speed reconstruction. The reconstructed image is applied as an input to a computer 936, which stores the image in a mass storage device 938. (One or more of the DAS 932, computer 936, etc. may be incorporated into a control module such as control module 240.)

Moreover, the computer 936 may also receive commands and scanning parameters from an operator via operator console 940 that may have an input device such as a keyboard (not shown in FIG. 9). An associated display 942 allows the operator to observe the reconstructed image and other data from the computer 936. Commands and parameters supplied by the operator are used by the computer 936 to provide control and signal information to the DAS 932 and the X-ray controller 928. Additionally, the computer 936 may operate a paddle controller 944, which controls the positioning of paddles or plates (e.g. upper plate 214 and lower plate 216) for compression and positioning of an object (e.g., breast 202) to be scanned by X-ray.

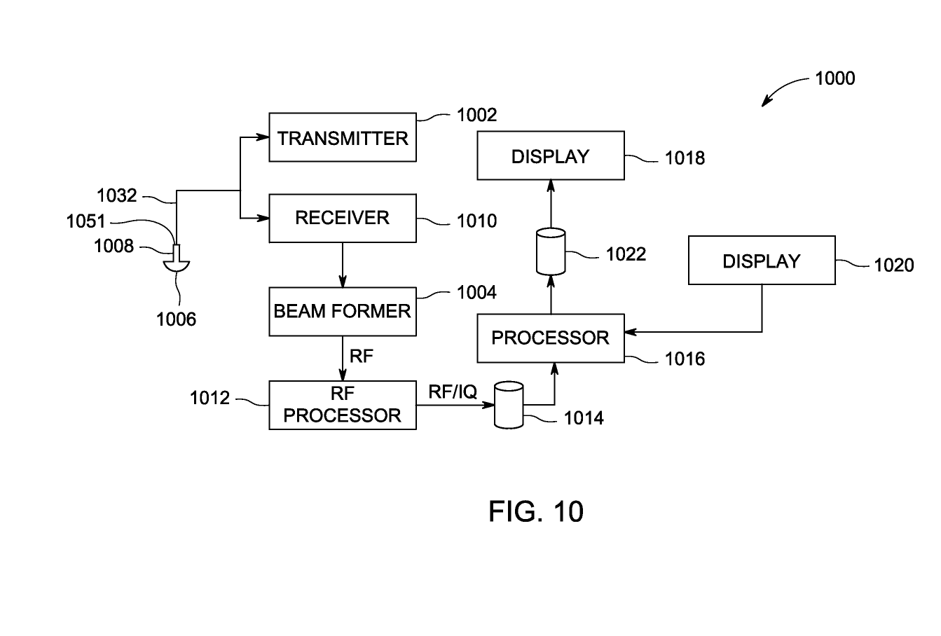

FIG. 10 is a block diagram of an exemplary ultrasound imaging system 1000 that is constructed in accordance with various embodiments. The ultrasound system 1000 is capable of electrical or mechanical steering of a soundbeam (such as in 3D space) and is configurable to acquire information (e.g., image slices) corresponding to a plurality of 2D representations or images of a region of interest (ROI) in a subject or patient, which may be defined or adjusted as described in more detail herein. The ultrasound system 1000 is configurable to acquire 2D images in one or more planes of orientation.

The ultrasound system 1000 includes a transmitter 1002 that, under the guidance of a beamformer 1004, drives an array of elements 1006 (e.g., piezoelectric elements) within a probe 1008 to emit pulsed ultrasonic signals, i.e. sound waves, into a body. For example, a probe such as the probe 1008 may be utilized as the ultrasound transducer 232. A variety of geometries may be used. As shown in FIG. 10, the probe 1008 may be coupled to the transmitter 1002 via the system cable 1032 and the connector 1051. The sound waves are back-scattered from structures in the body to produce echoes that return to the elements 1006. The echoes are received by a receiver 1010. The received echoes are passed through the beamformer 1004, which performs receive beamforming and outputs an RF signal. The RF signal then passes through an RF processor 1012. Optionally, the RF processor 1012 may include a complex demodulator (not shown) that demodulates the RF signal to form IQ data pairs representative of the echo signals. The RF or IQ signal data may then be routed directly to a buffer 1014 for storage.

In the above-described embodiment, the beamformer 1004 operates as a transmit and receive beamformer. Optionally, the probe 1008 includes a 2D array with sub-aperture receive beamforming inside the probe 1008. The beamformer 1004 may delay, apodize and/or sum each electrical signal with other electrical signals received from the probe 1008. The summed signals represent echoes from the ultrasound beams or lines. The summed signals are output from the beamformer 1004 to the RF processor 1012. The RF processor 1012 may generate different data types, e.g. B-mode, color Doppler (velocity/power/variance), tissue Doppler (velocity), and Doppler energy, for multiple scan planes or different scanning patterns. The RF processor 1012 gathers the information (e.g. I/Q, B-mode, color Doppler, tissue Doppler, and Doppler energy information) related to multiple data slices and stores the data information, which may include time stamp and orientation/rotation information, in the buffer 1014.

The ultrasound system 1000 also includes a processor 1016 (the processor 1016, for example, may be incorporated into a control module such as control module 240) to process the acquired ultrasound information (e.g., RF signal data or IQ data pairs) and prepare frames of ultrasound information for display on a display 1018. The processor 1016 is adapted to perform one or more processing operations according to a plurality of selectable ultrasound modalities on the acquired ultrasound data. Acquired ultrasound data may be processed and displayed in real-time during a scanning session as the echo signals are received. Additionally or alternatively, the ultrasound data may be stored temporarily in the buffer 1014 during a scanning session and then processed and displayed in an off-line operation.

The processor 1016 is connected to a user interface 1020 that may control operation of the processor 1016 as explained below in more detail. The display 1018 may include one or more monitors that present patient information, including diagnostic ultrasound images to the user for diagnosis and analysis. The buffer 1014 and/or a memory 1022 may store two-dimensional (2D) or three-dimensional (3D) data sets of the ultrasound data, where such 2D and 3D data sets are accessed to present 2D (and/or 3D images). The images may be modified and the display settings of the display 1018 may also be manually adjusted using the user interface 1020.

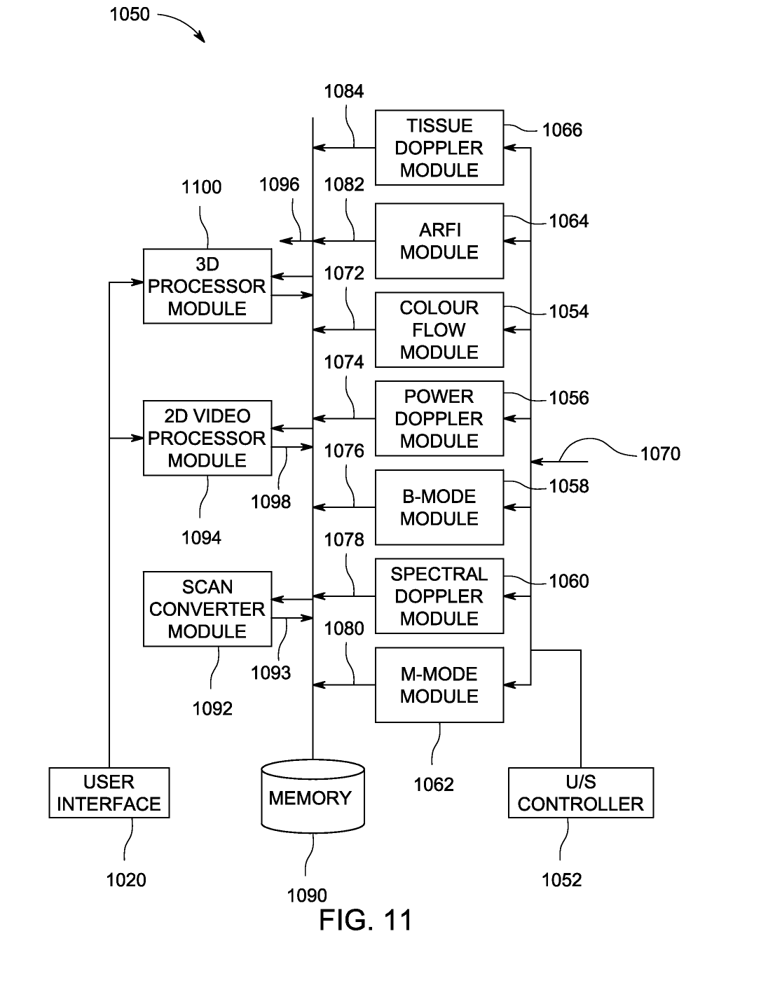

The various components of the ultrasound system 1000 may have different configurations. For example, FIG. 11 illustrates an exemplary block diagram of an ultrasound processor module 1050, which may be embodied as a portion of the processor 1016 shown in FIG. 10. The ultrasound processor module 1050 is illustrated conceptually as a collection of sub-modules, but may be implemented utilizing any combination of dedicated hardware boards, DSPs, processors, etc. Alternatively, the sub-modules of FIG. 11 may be implemented utilizing an off-the-shelf PC with a single processor or multiple processors, with the functional operations distributed between the processors. As a further option, the sub-modules of FIG. 11 may be implemented utilizing a hybrid configuration in which certain modular functions are performed utilizing dedicated hardware, while the remaining modular functions are performed utilizing an off-the shelf PC and the like. The sub-modules also may be implemented as software modules within a processing unit.

The operations of the sub-modules illustrated in FIG. 11 may be controlled by a local ultrasound controller 1052 or by the processor 1016. The sub-modules 1054-1066 perform mid-processor operations. The ultrasound processor module 1050 may receive ultrasound data 1070 in one of several forms. In the embodiment of FIG. 711, the received ultrasound data 1070 constitutes I,Q data pairs representing the real and imaginary components associated with each data sample. The I,Q data pairs are provided to one or more of a color-flow sub-module 1054, a power Doppler sub-module 1056, a B-mode sub-module 1058, a spectral Doppler sub-module 1060 and an M-mode sub-module 1062. Optionally, other sub-modules may be included such as an Acoustic Radiation Force Impulse (ARFI) sub-module 1064 and a Tissue Doppler (TDE) sub-module 1066, among others.

Each of sub-modules 1054-1066 are configured to process the I,Q data pairs in a corresponding manner to generate color-flow data 1072, power Doppler data 1074, B-mode data 1076, spectral Doppler data 1078, M-mode data 1080, ARFI data 1082, and tissue Doppler data 1084, all of which may be stored in a memory 1090 (or memory 1014 or memory 1022 shown in FIG. 10) temporarily before subsequent processing. For example, the B-mode sub-module 1058 may generate B-mode data 1076 including a plurality of B-mode image planes.

The data 1072-1084 may be stored, for example, as sets of vector data values, where each set defines an individual ultrasound image frame. The vector data values are generally organized based on the polar coordinate system.

A scan converter sub-module 1092 accesses and obtains from the memory 1090 the vector data values associated with an image frame and converts the set of vector data values to Cartesian coordinates to generate an ultrasound image frame 1093 formatted for display. The ultrasound image frames 1093 generated by the scan converter module 1092 may be provided back to the memory 1090 for subsequent processing or may be provided to the memory 1014 or 1022.

Once the scan converter sub-module 1092 generates the ultrasound image frames 1093 associated with, for example, B-mode image data, and the like, the image frames 1093 may be restored in the memory 1090 or communicated over a bus 1096 to a database (not shown), the memory 1014, and the memory 1022 and/or to other processors.

The scan converted data may be converted into an X,Y format for video display to produce ultrasound image frames. The scan converted ultrasound image frames are provided to a display controller (not shown) that may include a video processor that maps the video to a grey-scale mapping for video display. The grey-scale map may represent a transfer function of the raw image data to displayed grey levels. Once the video data is mapped to the grey-scale values, the display controller controls the display 1018 (shown in FIG. 10), which may include one or more monitors or windows of the display, to display the image frame. The image displayed in the display 1018 is produced from image frames of data in which each datum indicates the intensity or brightness of a respective pixel in the display.

Referring again to FIG. 11, a 2D video processor sub-module 1094 combines one or more of the frames generated from the different types of ultrasound information. For example, the 2D video processor sub-module 1094 may combine a different image frames by mapping one type of data to a grey map and mapping the other type of data to a color map for video display. In the final displayed image, color pixel data may be superimposed on the grey scale pixel data to form a single multi-mode image frame 1098 (e.g., functional image) that is again re-stored in the memory 1090 or communicated over the bus 1096. Successive frames of images may be stored as a cine loop, for example in the memory 1090. The cine loop represents a first in, first out circular image buffer to capture image data that is displayed to the user. The user may freeze the cine loop by entering a freeze command at the user interface 1020. The user interface 1020 may include, for example, a keyboard and mouse and all other input controls associated with inputting information into the ultrasound system 1000 (shown in FIG. 10).

A 3D processor sub-module 1100 is also controlled by the user interface 1020 and accesses the memory 1090 to obtain 3D ultrasound image data and to generate three dimensional images, such as through volume rendering or surface rendering algorithms as are known. The three dimensional images may be generated utilizing various imaging techniques, such as ray-casting, maximum intensity pixel projection and the like.

Thus, embodiments provide systems and methods wherein an electron beam size and focal spot size associated with an X-ray system may be adjusted. For example, a size of an electron beam may be reduced to provide a high resolution focal spot. Also, the size of an electron beam for a given X-ray tube assembly may be varied or adjusted by an operator of a scanning device or system including the X-ray tube, allowing one scanning device or system to perform a variety of scans using different resolution focal spots. Thus, some embodiments provide for improved adjustability of electron beam sizes, and/or improved resolution, for example, for X-ray imaging.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optical drive, and the like. The storage device may also be other similar means for loading computer programs or other instructions into the computer or processor.

As used herein, the term “computer”, “controller”, and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program. The individual components of the various embodiments may be virtualized and hosted by a cloud type computational environment, for example to allow for dynamic allocation of computational power, without requiring the user concerning the location, configuration, and/or specific hardware of the computer system.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments without departing from their scope. While the dimensions and types of materials described herein are intended to define the parameters of the various embodiments, they are by no means limiting and are merely exemplary. Many other embodiments will be apparent to those of skill in the art upon reviewing the above description. The scope of the various embodiments should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or the examples include equivalent structural elements with insubstantial differences from the literal languages of the claims.

What is claimed is:

1. An imaging assembly comprising:
a paddle assembly comprising first and second plates that are articulable with respect to each other, the first and second plates configured to receive and compress an object to be imaged;
an X-ray detection unit mounted proximate to at least one of the first and second plates;
an ultrasound module configured to acquire ultrasound information of the object to be imaged, the ultrasound module comprising an ultrasound transducer articulably mounted to at least one of the first and second plates, wherein the ultrasound transducer may be positioned to acquire ultrasound information of a selected portion of the object to be imaged; and
a control module configured to position the ultrasound transducer to scan a region of interest identified using X-ray information received from the X-ray detection unit while not scanning at least a portion of the object outside of the region of interest.

2. The assembly of claim 1, wherein the first plate is configured as an upper plate and the second plate is configured as a lower plate, and wherein the ultrasound transducer is configured to be articulable along an upper surface of the upper plate.

3. The assembly of claim 1, wherein the ultrasound module comprises a dispensing module comprising a reservoir, the dispensing module configured to dispense a material from the reservoir to acoustically couple the ultrasound transducer with a surface of at least one of the first and second plates.

4. The assembly of claim 1, wherein the ultrasound transducer comprises an array of transducers configured to be articulated in a substantially lateral direction across a surface of at least one of the first and second plates.

5. The assembly of claim 1, wherein the first and second plates are articulable relative to each other in a compression direction and the ultrasound transducer is articulable in an ultrasound direction, and wherein the compression direction and the ultrasound direction are substantially perpendicular to each other.

6. The assembly of claim 1, wherein the control module is configured to autonomously select the region of interest.

7. The assembly of claim 6, wherein the control module is configured to autonomously release the first and second plates from a compression position if no region of interest is identified.

8. The assembly of claim 1, further comprising an interface module operably connected to the control module, the interface module configured to display information corresponding to an X-ray scan to a practitioner, receive a selection of the region of interest from the practitioner, and provide information corresponding to the region of interest to the control module.

9. The assembly of claim 1, wherein the control module is configured to determine an estimated time for performing an ultrasound scan of the region of interest.

10. The assembly of claim 9, wherein the control module is configured to reduce an amount of compression on the object between the first and second plates if the estimated time exceeds a threshold.

11. The assembly of claim 1, further comprising a housing configured to retain a material through which the ultrasound transducer passes, the material configured to acoustically couple the ultrasound transducer with a surface of at least one of the first and second plates.

12. The assembly of claim 11, wherein the housing includes a port configured to allow removal of at least a portion of the material from the housing during an X-ray scan.

13. The assembly of claim 11, wherein the housing defines a sealed volume, wherein the ultrasound transducer is disposed within the sealed volume, wherein the ultrasound transducer is articulated via an actuator, and wherein at least a portion of the actuator is disposed outside of the sealed volume.

14. The assembly of claim 1, wherein the ultrasound assembly is configured to be positioned outside of a field of view of an X-ray source configured to provide an X-ray beam to the X-ray detection unit when the object is being subjected to an X-ray.

15. A method for imaging an object comprising:
obtaining an X-ray image of the object using information acquired by the X-ray detection unit, wherein the object has been compressed between first and second plates, wherein at least one of the first and second plates has an X-ray detection unit associated therewith;
identifying a region of interest of the object; and
selectively ultrasound imaging the region of interest of the object while the object remains under compression while not ultrasound imaging at least a portion of the object outside of the region of interest.

16. The method of claim 15, wherein the region of interest is identified autonomously by a control module.

17. The method of claim 15, wherein the object is released from between the first and second plates and re-positioned for a second scan after the selectively ultrasound imaging the region of interest.

18. The method of claim 15, further comprising estimating, at a control module, an ultrasound time corresponding to an estimated time for selectively ultrasound scanning the region of interest, and reducing an amount of compression on the object between the first and second plates based on the ultrasound time if the ultrasound time exceeds a threshold.

19. The method of claim 15, further comprising reducing an amount of compression on the object between the first and second plates after the obtaining an X-ray image and before the selectively ultrasound scanning the region of interest.

20. The method of claim 15, wherein the step of selectively ultrasound scanning the region of interest comprises positioning an ultrasound transducer by articulating the ultrasound transducer across a surface of at least one of the first and second plates.

21. The method of claim 20, further comprising dispensing a liquid from an ultrasound module to acoustically couple the ultrasound transducer and the surface of the at least one of the first and second plates.

22. The method of claim 20, wherein the ultrasound transducer is articulated through a housing configured to retain a material configured to acoustically couple the ultrasound transducer and a surface of the housing.

23. The method of claim 22, further comprising evacuating at least a portion of the material from the housing during the obtaining of the X-ray image of the object.

24. A tangible and non-transitory computer readable medium comprising one or more computer software modules configured to direct a processor to:
obtain an X-ray image of an object using information acquired by the X-ray detection unit, wherein the object has been compressed between first and second plates, wherein at least one of the first and second plates has an X-ray detection unit associated therewith;
control an ultrasound module to selectively ultrasound image the region of interest of the object while the object remains under compression while not ultrasound imaging at least a portion of the object outside of the region of interest.

25. The tangible and non-transitory computer readable medium of claim 24, wherein the one or more software modules are further configured to direct the processor to autonomously identify the region of interest.

26. The tangible and non-transitory computer readable medium of claim 24, wherein the one or more software modules are further configured to direct the processor to estimate an ultrasound time corresponding to an estimated time for selectively ultrasound imaging the region of interest, and reducing an amount of compression on the object between the first and second plates based on the ultrasound time if the ultrasound time exceeds a threshold.

27. The tangible and non-transitory computer readable medium of claim 24, wherein the one or more software modules are further configured to direct the processor to reduce an amount of compression on the object between the first and second plates after obtaining an X-ray image and before selectively ultrasound scanning the region of interest.

28. The tangible and non-transitory computer readable medium of claim 24, wherein the one or more software modules are further configured to direct the processor to position an ultrasound transducer by articulating the ultrasound transducer across a surface of at least one of the first and second plates.

29. The tangible and non-transitory computer readable medium of claim 28, wherein the one or more software modules are further configured to direct the processor to dispense a liquid from an ultrasound module to acoustically couple the ultrasound transducer and the surface of the at least one of the first and second plates.
